(19) United States
(12) Patent Application Publication
(10) Pub. No.: US 2020/0359950 A1
(43) Pub. Date: Nov. 19, 2020

(54) BLOOD-COLLECTING DEVICE, BLOOD-COLLECTING TUBE USABLE FOR BLOOD-COLLECTING DEVICE, AND PRODUCTION METHOD OF BLOOD-COLLECTING DEVICE

(71) Applicant: Sunspots Co., Ltd., Tanabe-shi (JP)

(72) Inventor: Shin TAIRA, Tanabe-shi (JP)

(73) Assignee: Sunspots Co., Ltd.

(21) Appl. No.: 16/601,048

(22) Filed: Oct. 14, 2019

(30) Foreign Application Priority Data
May 15, 2019 (JP) 2019-091821

(51) Int. Cl. A61B 5/15

(52) U.S. Cl. CPC A61B 5/150206; A61B 5/150374; A61B 5/150801; A61B 5/150343

(57) ABSTRACT

There are provided a blood-collecting device by which a blood-collecting operation can be performed by single hand and the blood can be collected in a short time without a risk of hemolysis, and a blood-collecting tube usable for blood-collecting device. A blood-collecting tube, of which a tip portion is housed in a tube holding portion and which accommodates the blood coming out of a base end side of a needle tube, includes a tube structure opened at both ends, and a tube attached to the tube structure. A tip portion of the tube structure is housed in a hollow portion of a tube holding portion of a needle base. The tube preferably has elasticity. The tube is attached to the tube structure using elastic deformation of the tube, allowing the tube to be in close contact with an inner circumferential surface of the needle base.

### TECHNICAL FIELD

The present disclosure relates to a blood-collecting device, blood-collecting tube usable for blood-collecting device, and production method of blood-collecting device.

As a conventional blood-collecting method used for collecting the blood from a human body or an animal, an injection syringe method has been known in which a blood-collecting needle is inserted in a blood-collecting site and the blood is suctioned by a piston or the like and collected in an injection syringe (for example, Patent literature 1).

### CITATION LIST

### Patent Literature

Patent Literature 1: Japanese Patent Application Laid-Open No. 05-300939.

### SUMMARY

### Technical Problem

However, the injection syringe method requires an insertion operation of the blood-collecting needle and a suction operation of the piston, and thus the blood needs to be collected by both hands. Further, increasing a suction pressure causes a problem of inducing severe hemolysis. The hemolysis problem becomes more prominent as the blood-collecting needle becomes finer, and thus the problem cannot be ignored in an animal having a small blood vessel. On the other hand, when a suction pressure is reduced not to induce hemolysis, it takes time to suction the blood into the injection syringe, causing a problem of requiring a longer time for collecting the blood. Further, when the blood is collected from a subject having difficulty in communication such as an animal and a newborn baby, it is difficult to keep the subject still during the blood collection.

Under such a circumstance, the present inventor made intensive studies and found a blood-collecting device allowing a blood-collecting operation with single hand without a risk of hemolysis and a blood-collecting tube usable for the blood-collecting device.

The present disclosure has been made in view of the above circumstance and has its object to provide a blood-collecting device by which a blood-collecting operation can be performed by single hand and the blood can be collected in a short time without a risk of hemolysis, a blood-collecting tube usable for the blood-collecting device, and a production method of the blood-collecting device.

### Solution to Problem

Owing to the earnest investigation made by the present inventor, the above-described object is achieved by the following means.

A blood-collecting tube of the present disclosure includes a tube structure opened at both ends, a projecting structure projecting from a tube circumferential surface of the tube structure, wherein: the tube structure has translucency; the projecting structure is an elastic body; the projecting structure is positioned at an end portion of the tube structure on one side; and the tube structure and the projecting structure can be inserted in a hollow portion of a needle base that supports a blood-collecting needle.

A blood-collecting device of the present disclosure includes a blood-collecting tube; and a blood-collecting needle unit to which the blood-collecting tube can be attached.

A method for producing a blood-collecting device of the present disclosure includes an insertion step of inserting the projecting structure and the tube structure in a hollow portion of the needle base; and a push-in step of pushing the projecting structure and the tube structure in the hollow portion of the needle base.

### Advantageous Effects of Invention

According to the present disclosure, it becomes possible to provide a blood-collecting device by which a blood-collecting operation can be performed by single hand and the blood can be collected in a short time without a risk of hemolysis, a blood-collecting tube usable for the blood-collecting device, and a production method of the blood-collecting device.

### BRIEF DESCRIPTION OF DRAWINGS

FIG. 1 is an elevation view illustrating an outline of a first blood-collecting device (the first embodiment of the blood-collecting device);

FIG. 2 is an elevation view illustrating outlines of a blood-collecting needle unit and a blood-collecting tube;

FIGS. 3A and 3B are each an elevation view illustrating outlines of the blood-collecting needle unit and the blood-collecting tube;

FIG. 4A is an exploded view illustrating an outline of the blood-collecting tube, FIG. 4B is an explanatory view illustrating an outline of an opening end surface of a tube structure, and FIG. 4C is an explanatory view illustrating an outline of an opening end surface of a tube;

FIG. 5 is an explanatory view illustrating an outline of how to hold the blood-collecting tube;

FIG. 6 is an explanatory view illustrating an outline of how to hold the blood-collecting device;

FIGS. 7A and 7B are each an explanatory view illustrating an outline of a second blood-collecting device (the second embodiment of the blood-collecting device); and

FIGS. 8A and 8B are each an explanatory view illustrating an outline of a third blood-collecting device (the third embodiment of the blood-collecting device).

### DESCRIPTION OF EMBODIMENTS

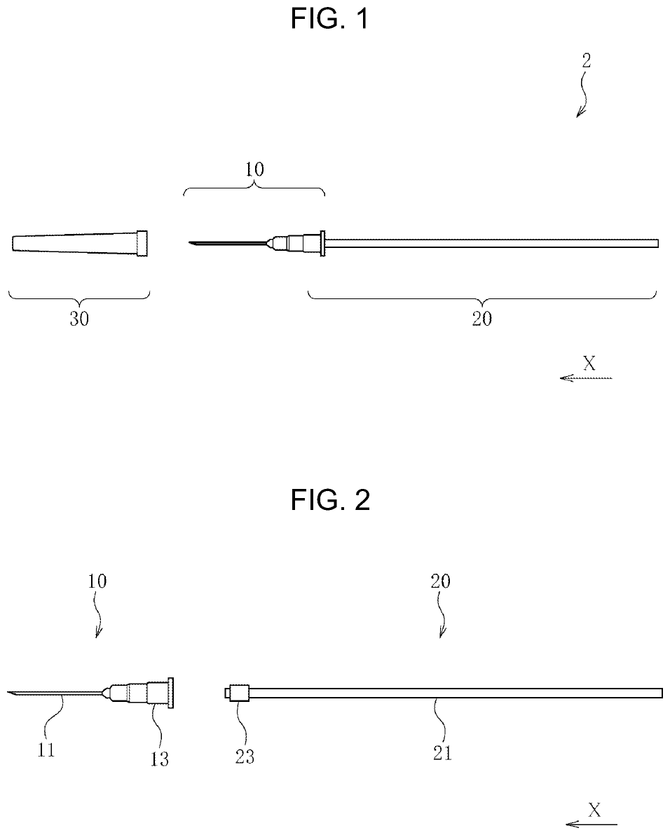

As shown in FIGS. 1 and 2, a blood-collecting device 2 is used for collecting the blood from human or an animal and includes a blood-collecting needle unit 10, a blood-collecting tube 20 detachable from a base end side (right side in the drawings) of the blood-collecting needle unit 10, and a cap 30 detachable from a tip side (left side in the drawings) of the blood-collecting needle unit 10.

The blood-collecting needle unit 10 includes a needle tube 11 of which a tip portion is inserted in a blood vessel and a needle base 13 for supporting a base end side of the needle tube 11.

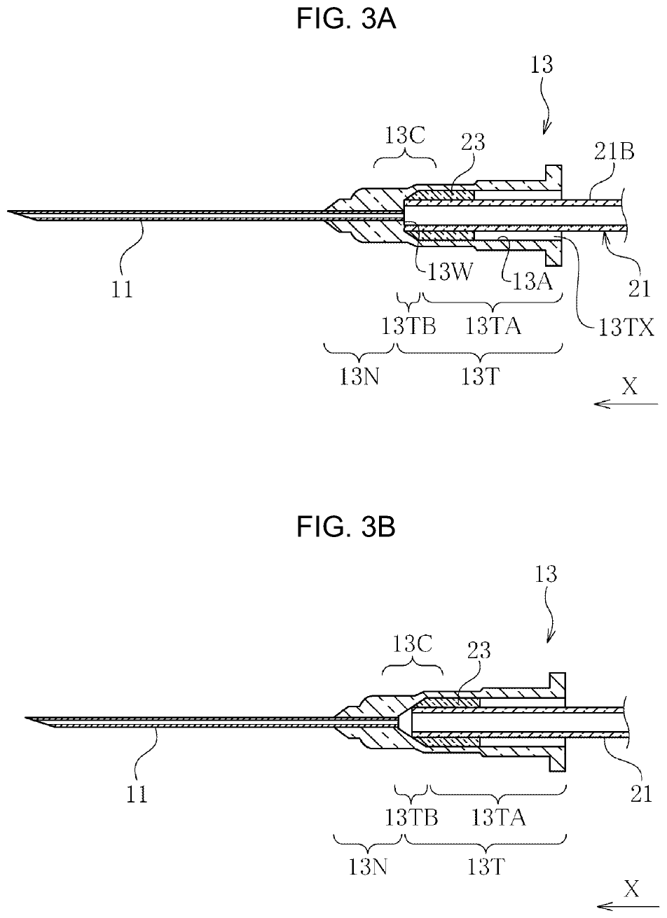

As shown in FIGS. 2, 3A and 3B, the needle tube 11 includes a tip portion to be inserted in a blood vessel and a base end portion from which the blood introduced through the tip portion comes out. The size of the needle tube 11 is not particularly limited as long as a function of the needle tube 11 is exhibited. However, for example, the inner diameter is preferably 0.05 mm or more and 2 mm or less and the outer diameter is preferably 0.1 mm or more and 2.5 mm or less.

The needle base 13 is formed in a cylindrical shape and includes a needle support portion 13N for holding the needle tube 11 provided on a tip side of the needle base 13, a tube holding portion 13T for holding a blood-collecting tube 20 provided on a base end side of the needle base 13, and a connecting portion 13C for connecting the needle support portion 13N and the tube holding portion 13T.

The needle support portion 13N and the tube holding portion 13T are each formed in a cylindrical shape having an axis in an X direction. The needle support portion 13N and the tube holding portion 13T are coaxially connected by the connecting portion 13C. The needle support portion 13N includes a hole capable of holding the needle tube 11. The needle tube 11 inserted in this hole is coaxially held with the needle base 13.

The tube holding portion 13T includes an equal diameter portion 13TA on a base end side and a reduced diameter portion 13TB on a tip side. The equal diameter portion 13TA has an equal inner diameter from the base end side to the tip side. The reduced diameter portion 13TB has a diameter becoming smaller from the base end side toward the tip side.

As a material for forming the needle base 13, plastic, glass, or the like can be used. Further, the needle base 13 preferably has translucency and is also preferably colorless and transparent. This makes it possible to visually confirm whether the blood exists inside the needle base 13.

As shown in FIG. 1, the cap 30 is made attachable to and detachable from the tip side of the needle base 13. When the cap 30 is attached to the tip side of the needle base 13, the cap 30 covers the needle tube 11.

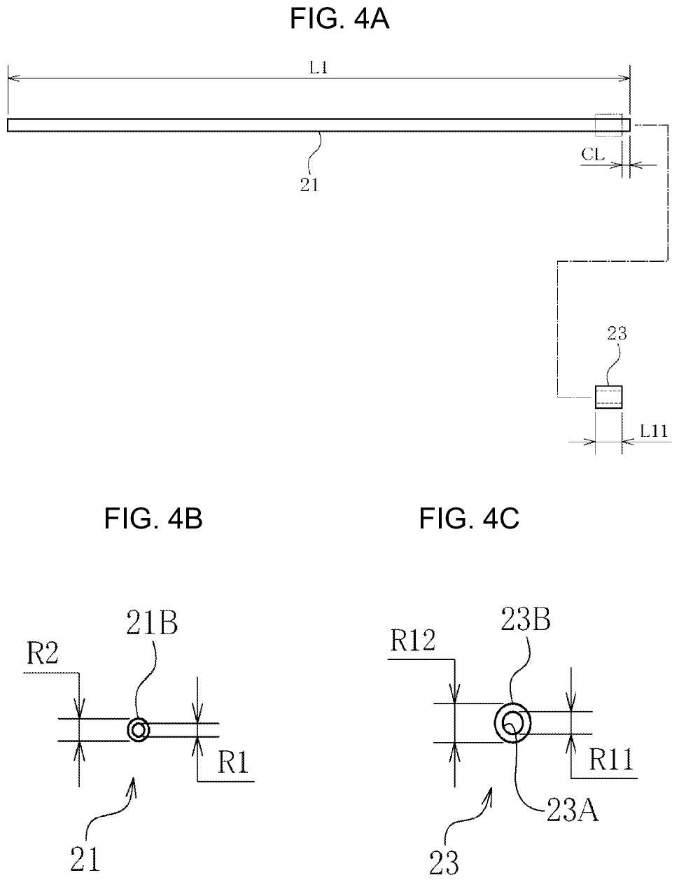

As shown in FIGS. 2 to 4C, the blood-collecting tube 20 is formed in a cylindrical shape and a tip portion thereof is housed in the tube holding portion 13T. The blood-collecting tube 20 accommodates, in a hollow portion thereof, the blood coming out of the base end side of the needle tube 11. The blood-collecting tube 20 includes a tube structure 21, which is opened at both ends, for accommodating the blood, and a tube 23 attached to the tube structure 21.

A tip portion of the tube structure 21 is housed in a hollow portion 13TX of the tube holding portion 13T of the needle base 13. The size of the tube structure 21 is not particularly limited as long as the tube structure 21 can be housed in the tube holding portion 13T. As the size of the tube structure 21, the size of the needle tube 11, an inner diameter R1, and a length L1 may be determined according to an amount of the blood desired to be collected. An outer diameter R2 only needs to be smaller than or equal to an inner diameter of the needle base. Further, as a concrete numeric value, for example, the inner diameter R1 is preferably 1 mm or more and 3 mm or less, while the outer diameter R2 is preferably 2 mm or more and 5 mm or less. The length L1 of the tube structure 21 is preferably 50 mm or more and 200 mm or less. This configuration is not affected by a diameter of the needle tube 11, and thus a larger amount of the blood (for example, 0.1 mL to 0.5 mL) can be collected while using a fine blood-collecting needle.

The tube structure 21 preferably has translucency and is also preferably colorless and transparent. This makes it possible to visually confirm whether the blood exists inside the tube structure 21.

As a material for forming the tube structure 21, plastic, glass, or the like can be used. Plastic usable as the material for forming the tube structure 21 may be either a thermosetting resin or a thermoplastic resin, and examples thereof include polyethylene, polypropylene, polyethylene terephthalate (PET-G), polyvinyl chloride, polyvinylidene chloride, an EVA resin, polystyrene, an AS resin, an ABS resin, an acrylic resin, polyamide, polytetrafluoroethylene, an ethylene-vinyl acetate copolymer, polycarbonate, a phenol resin, a melamine resin, unsaturated polyester, and an epoxy resin. Further, the tube structure 21 may be treated with lithium heparin.

The tube structure 21 preferably has higher rigidity than the tube 23. This makes the blood collection easy and allows an airtight state to be maintained.

The tube 23 is attached to the tube structure 21. An inner circumferential surface 23A of the tube 23 is preferably in close contact with an outer circumferential surface 21B of the tube structure 21. Further, an outer circumferential surface 23B of the tube 23 is preferably in close contact with an inner circumferential surface 13A of the needle base 13 in a state where the blood-collecting tube 20 is inserted in the needle base 13 (FIGS. 3A and 3B).

The tube 23 preferably has elasticity. Further, an inner diameter R11 of the tube 23 is preferably smaller than the outer diameter R2 of the tube structure 21. The tube 23 is attached to the tube structure 21 using elastic deformation of the tube 23, allowing the tube 23 to be in close contact with the inner circumferential surface 13A of the needle base 13. For example, the inner diameter R11 of the tube 23 is preferably 0.3 times or more and 0.99 times or less, more preferably 0.5 times or more and 0.95 times or less, of the outer diameter R2 of the tube structure 21. Further, an outer diameter R12 of the tube 23 is preferably more than one time and 1.5 times or less, more preferably 1.01 times or more and 1.3 times or less, of the inner diameter of the needle base 13. A length L11 of the tube 23 is preferably 3 mm or more and 5 mm or less. Although being speculative, one of merits of making the inner diameter R11 of the tube 23 smaller than the outer diameter R2 of the tube structure 21 is thought to be that “a balance between a friction force between the tube 23 and the tube structure 21 and a friction force between the tube 23 and the needle base 13 becomes desirable for performing an attaching/detaching operation of the blood-collecting tube 20 to/from the needle base 13”.

The tube structure 21 and the tube 23 are formed separately in this manner, and thus, when the blood-collecting tube 20 is inserted in the needle base 13, the tube 23 can be moved to a specified position in the tube structure 21 while the outer circumferential surface and inner circumferential surface of the tube 23 are kept in close contact with the inner circumferential surface 13A of the needle base 13 and the outer circumferential surface 21B of the tube structure 21, respectively. That is, the tube 23 can be moved on the tube structure 21 while maintaining an airtight state. In this configuration, when the blood-collecting tube 20 is inserted in the needle base 13, an airtight state can be created with a minimum insertion force. As a result, the blood-collecting tube 20 can also be removed from the needle base 13 with a minimum force.

The tube 23 preferably has translucency and is also preferably colorless and transparent. This makes it possible to visually confirm whether the blood exists inside the tube 23.

Examples of a material for forming the tube 23 include a silicone resin, a rubber, a plaster, a putty, and a clay. As the rubber, for example, either a natural rubber or a synthetic rubber may be used. Examples of the synthetic rubber include a diene rubber and a non-diene rubber. Examples of the synthetic rubber include an isoprene rubber I (IR), a butadiene rubber (BR), a styrene-butadiene rubber (SBR), a butyl rubber (IIR), a nitrile rubber (NBR), a chloroprene rubber (CR), an ethylene propylene rubber (EPM, EPDM), an acrylic rubber (ACR), a chlorosulfonated polyethylene rubber (CSM), a fluorine rubber (FKM), an epichlorohydrin rubber (CO, ECO), a urethane rubber (U), a silicone rubber, an ethylene-vinylacetate rubber, and a polysulfide rubber. Note that, as a material for forming the tube 23, a thermoplastic elastomer may be used. Examples of the thermoplastic elastomer include a styrene-based elastomer, an olefin-based elastomer, an ester-based elastomer, a urethane-based elastomer, an amide-based elastomer, a PVS-based elastomer, and a fluorine-based elastomer.

The tube 23 is preferably disposed in the vicinity of an opening portion of the tube structure 21 on the tip side. A distance CL from an end surface of the tube 23 on the tip side to an end surface of the tube structure 21 on the tip side is not particularly limited as long as it is within a range in which the effect of the present embodiment is exhibited. However, for example, an upper limit of the distance CL is preferably 3 mm or less, more preferably 2 mm or less. Further, the end surface of the tube 23 on the tip side may be flush with the end surface of the tube structure 21 on the tip side.

The blood-collecting device 2 includes the tube 23 attached to the tube structure 21. Further, a space between the tube structure 21 and the needle base 13 can be brought into an airtight state by elastic deformation of the tube 23. Thus, the blood is flown into the needle base 13 via the needle tube 11 immediately after the needle tube 11 is inserted in the blood vessel. Although being speculative, a phenomenon in which the blood is flown into the needle base 13 immediately after the insertion of the needle tube 11 is thought to be caused by the blood pressure of an animal or the like as a subject and a capillarity phenomenon. As described above, using the blood-collecting device 2 makes it possible to collect the blood without creating a negative pressure, unlike an injection syringe. Further, unlike an injection syringe, a suction operation of the piston is not required, allowing the blood collection with single hand. Further, even if the outer diameter of the blood-collecting tube 20 to be used is smaller than the inner diameter of the needle base 13, a gap between these diameters can be filled by the tube 23, making it possible to use a desired blood-collecting tube 20 and a generic blood-collecting needle unit 10. In this manner, the blood-collecting tube 20 can be selected according to the amount of the blood desired to be collected and attached to the generic blood-collecting needle unit 10, and thus the desired amount of the blood can be collected at low cost.

### EXAMPLES

The blood-collecting device 2 was produced by the following procedures.

The tube structure 21 in use was made of a transparent resin (polyethylene terephthalate PET-G) and had an inner diameter of 2.0 mm, an outer diameter of 3.0 mm, and a length of 110 mm. The tube 23 in use was made of a transparent resin (silicone resin) and had an inner diameter of 2.0 mm, an outer diameter of 3.5 mm, and a length of 5 mm. Next, the tube 23 was attached to the tube structure 21. The outer diameter of the tube 23 attached to the tube structure 21 was 4 mm. In this operation, the distance CL from the end surface of the tube 23 on the tip side to the end surface of the tube structure 21 on the tip side was set to 2 mm. The blood-collecting tube 20 was obtained in this manner.

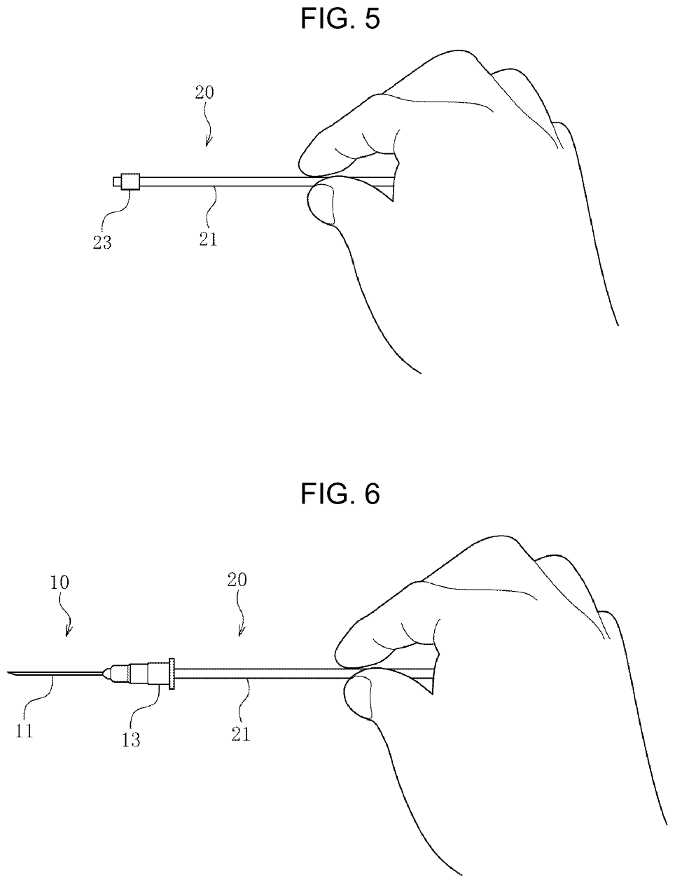

The blood-collecting needle unit 10 was prepared. The needle base 13 had a shape in which the diameter is reduced from a base end portion (inner diameter of 4.1 mm) toward a tip portion (inner diameter of 3.0 mm). The needle tube 11 had an inner diameter of 0.3 mm and an outer diameter of 0.55 mm. The tube structure 21 of the blood-collecting tube 20 thus obtained was held by fingers (FIG. 5) and pushed in a hollow portion of the needle base 13. The push-in operation was continued until the tip surface of the tube structure 21 abutted on an inner wall surface of the needle base 13 (FIG. 3A, 3B). During the push-in operation, the tube 23 attached to the tube structure 21 was introduced into the inside of the needle base 13 while being deformed by the inner circumferential surface of the needle base 13. In a state where the tip surface of the tube structure 21 abutted on an inner wall surface 13W of the needle base 13, the tube 23 was deformed so as to fill the gap between the tube structure 21 and the needle base 13. The blood-collecting device 2 was obtained in this manner.

The blood-collecting device 2 thus obtained was used to a dog (target). As shown in FIG. 6, the tube structure 21 was held by single hand and a blood-collecting needle was inserted in the hind limb lateral saphenous vein. Immediately after the insertion of the blood-collecting needle, the posture was maintained as shown in FIG. 6. In this manner, immediately after the insertion of the blood-collecting needle, the blood flown into the needle base 13 via the needle tube 11 could be visually confirmed. Three to five seconds after the insertion of the blood-collecting needle, the blood-collecting needle was removed from the hind limb lateral saphenous vein. The amount of the collected blood was about 0.3 mL.

After collecting the blood, the tube structure 21 was removed from the blood-collecting needle unit 10 while an opening of the tube structure 20 on the base end side was closed with a finger. There was little blood left in the needle base 13 after removing the tube structure 20, and thus it could be confirmed that the space between the tube structure 21 and the needle base 13 was in an airtight state by the tube 23.

In the above-described embodiment, the tube 23 was attached to the tube structure 21. However, the present disclosure is not limited to this configuration, and the tube 23 may be integrally formed with the tube structure 21.

In the above-described embodiment, the tube holding portion 13T included the equal diameter portion 13TA and the reduced diameter portion 13TB. However, the present disclosure is not limited to this configuration, and either the equal diameter portion 13TA or the reduced diameter portion 13TB may be omitted.

In the above-described embodiment, the tip surface of the tube structure 21 was inserted until it abutted on the inner wall surface 13W of the needle base 13 (FIG. 3A). However, the present disclosure is not limited to this configuration, and the tip surface of the tube structure 21 may be inserted until it abuts on the inner circumferential surface of the reduced diameter portion 13TB of the needle base 13 (FIG. 3B).

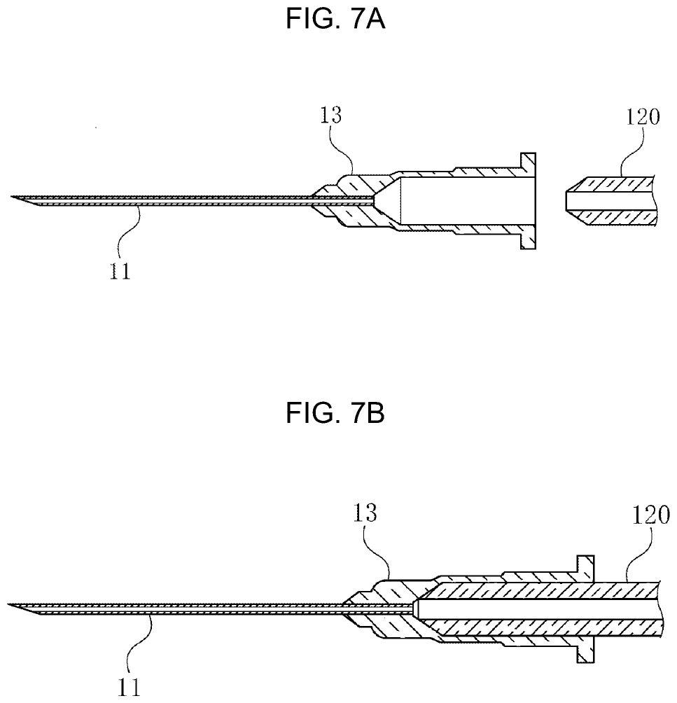

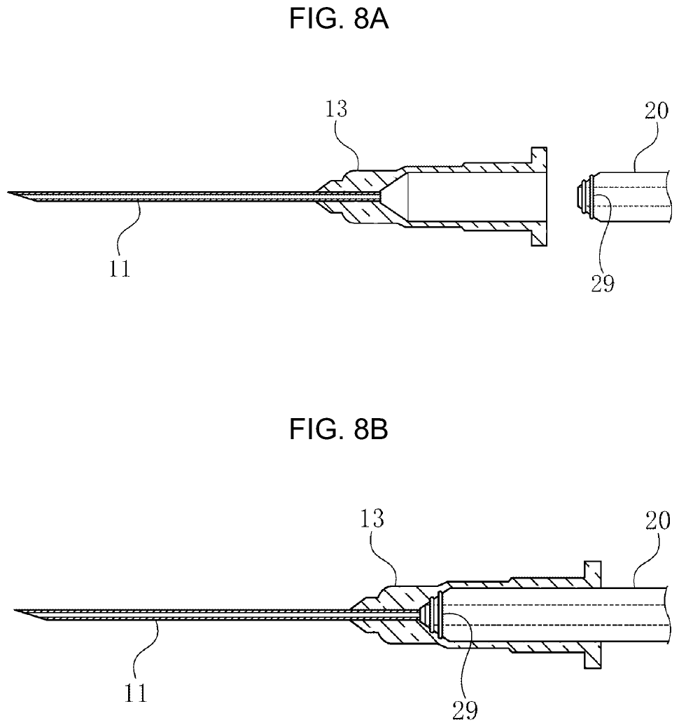

In the above-described embodiment, the blood-collecting tube 20 including the tube structure 21 and the tube 23 attached to the tube structure 21 was used. However, the present disclosure is not limited to this configuration, and a blood-collecting tube 120 having an external form that coincides with the shape of the inner circumferential surface of the needle base 13 may be used (FIGS. 7A and 7B). Further, although the blood-collecting tube 20 including the tube 23 attached to the tube structure 21 was used, the blood-collecting tube 20 including a projection 29 may be used (FIGS. 8A and 8B). The projection 29 is provided so as to project from the circumferential surface of the tube structure 21. The space between the tube structure 21 and the needle base 13 can be brought in to an airtight state by the projection 29.

Note that the present disclosure is not limited to the above-described embodiments, and, needless to say, various modifications may be made without departing from the gist of the present disclosure.

### REFERENCE SINGS LIST

2 blood-collecting device
10 blood-collecting needle unit
11 needle tube
13 needle base
13A inner circumferential surface
13C connecting portion
13N needle support portion
13T tube holding portion
13TA equal diameter portion
13TB reduced diameter portion
13TX hollow portion
13W inner wall surface
20 blood-collecting tube
21 tube structure
21B outer circumferential surface
23 tube
23A inner circumferential surface
23B outer circumferential surface
29 projection
30 cap
120 blood-collecting tube

What is claimed is:

1. A blood-collecting tube comprising:
a tube structure opened at both ends; and
a projecting structure projecting from a tube circumferential surface of the tube structure, wherein:
the tube structure has translucency;
the projecting structure is an elastic body;
the projecting structure is positioned at an end portion of the tube structure on one side; and
the tube structure and the projecting structure can be inserted in a hollow portion of a needle base that supports a blood-collecting needle.

2. The blood-collecting tube according to claim 1, wherein
the projecting structure includes a cylindrical body, and
a hollow portion of the cylindrical body is an insertion hole for the tube structure.

3. The blood-collecting tube according to claim 2, wherein the projecting structure is in close contact with the tube structure.

4. The blood-collecting tube according to claim 3, wherein the projecting structure is freely movable with respect to the tube structure.

5. The blood-collecting tube according to claim 4, wherein a diameter of the insertion hole is smaller than an outer diameter of the tube structure.

6. The blood-collecting tube according to claim 1, wherein the projecting structure has translucency.

7. The blood-collecting tube according to claim 6, wherein the projecting structure is made of a silicone resin.

8. The blood-collecting tube according to claim 1, wherein the projecting structure is integrally formed with the tube structure.

9. A blood-collecting device comprising:
a blood-collecting tube; and
a blood-collecting needle unit to which the blood-collecting tube can be attached, wherein:
the blood-collecting tube includes
a tube structure opened at both ends, and
a projecting structure projecting from a tube circumferential surface of the tube structure;
the tube structure has translucency;
the projecting structure is an elastic body;
the blood-collecting needle unit includes
a blood-collecting needle, and
a needle base formed in a cylindrical shape, the needle base being configured to support an end portion of the blood-collecting needle on one side;
an end portion of the tube structure on one side and the projecting structure positioned at the end portion of the tube structure on the one side are inserted in a hollow portion of the needle base; and
the projecting structure fills a gap between the circumferential surface of the tube structure and the needle base.

10. The blood-collecting device according to claim 9, wherein
the projecting structure includes a cylindrical body, and
a hollow portion of the cylindrical body is an insertion hole for the tube structure.

11. The blood-collecting device according to claim 10, wherein the projecting structure is in close contact with the tube structure.

12. The blood-collecting device according to claim 11, wherein the projecting structure is freely movable with respect to the tube structure.

13. The blood-collecting device according to claim 12, wherein a diameter of the insertion hole is smaller than an outer diameter of the tube structure.

14. The blood-collecting device according to claim 9, wherein the projecting structure has translucency.

15. The blood-collecting device according to claim 14, wherein the projecting structure is made of a silicone resin.

16. A method for producing a blood-collecting device,
the blood-collecting device including
a blood-collecting tube, and
a blood-collecting needle unit to which the blood-collecting tube can be attached;
the blood-collecting tube including
a tube structure opened at both ends, and
a projecting structure projecting from a tube circumferential surface of the tube structure;
the tube structure having translucency;
the projecting structure being an elastic body;
the blood-collecting needle unit including
a blood-collecting needle, and
a needle base formed in a cylindrical shape, the needle base being configured to support an end portion of the blood-collecting needle on one side,
the method comprising:
an insertion step of inserting the projecting structure and the tube structure in a hollow portion of the needle base; and
a push-in step of pushing the projecting structure and the tube structure in the hollow portion of the needle base, wherein
in the insertion step, the projecting structure is elastically deformed by causing the projecting structure to come into contact with the needle base, and
in the push-in step, the elastically deformed projecting structure fills in a gap between the circumferential surface of the tube structure and an inner circumferential surface of the needle base.

17. The method for producing a blood-collecting device according to claim 16, further comprising an abutting step of causing the tube structure to abut to an inner wall surface of the needle base, wherein,
in the abutting step, the elastically deformed projecting structure fills in the gap between the circumferential surface of the tube structure and the inner circumferential surface of the needle base.

18. The method for producing a blood-collecting device according to claim 17, wherein
the projecting structure includes a cylindrical body,
a hollow portion of the cylindrical body is an insertion hole for the tube structure,
the method further comprises a blood-collecting tube-side attachment step of inserting the tube structure in the insertion hole for the tube structure, the blood-collecting tube-side attachment step being performed before the insertion step,
in the blood-collecting tube-side attachment step, the projecting structure is elastically deformed by causing the projecting structure to come into contact with the tube structure, and
the projecting structure is in close contact with tube structure.
